(19) United States
(12) Patent Application Publication
(10) Pub. No.: US 2026/0284183 A1
(43) Pub. Date: Sep. 24, 2026

(54) THERAPEUTIC FOOD PRODUCTS FOR FOOD ALLERGY TREATMENT

(71) Applicant: Stephanie WIBOM, Dallas, TX (US)

(72) Inventor: Stephanie WIBOM, Dallas, TX (US)

(21) Appl. No.: 19/681,890

(22) Filed: May 19, 2026

Related U.S. Application Data
(63) Continuation of application No. 19/474,856, filed on Apr. 12, 2024.
(60) Provisional application No. 63/495,949, filed on Apr. 13, 2023.

(51) Int. Cl. A61K 39/35; A23L 15/10; A23L 25/00; A23L 25/10; A61K 39/00

(52) U.S. Cl. CPC A61K 39/35; A23L 15/10; A23L 25/10; A23L 25/30; A61K 2039/577

(57) ABSTRACT

A food product composition includes an active ingredient comprising a food allergen protein and one or more inactive ingredients comprising food-based ingredients. The food product requires no mixing prior to consumption, and the food product is substantially free of anti-caking agents and filling agents. The food product comprises a predefined dosage of the active ingredient, and the predefined dosage is within about 10% of a stated weight of the food allergen protein.

### CROSS-REFERENCE TO RELATED APPLICATIONS

The present application is a continuation of U.S. application Ser. No. 19/474,856 filed on Oct. 13, 2025, which is the U.S. National Phase Application of PCT/US2024/024414 filed on Apr. 12, 2024, which claims priority to U.S. Provisional Application 63/495,949 filed on Apr. 13, 2023 and entitled “THERAPEUTIC FOOD PRODUCTS FOR FOOD ALLERGY IMMUNOTHERAPY,” all of which are incorporated herein by reference in their entirety.

### BACKGROUND

Humans can be allergic to various foods or proteins in the foods, and in some instances, the allergies can be life threatening. A food allergy is an adverse immune response to a food allergen such as a food protein. The food allergies can occur based on the subject's immune system response to the food allergens. In order to help to alleviate or lessen the effects of the food allergies, a subject can be systematically exposed to the food allergens over time. The exposure can lessen the effects of the specific food allergies, thereby reducing the risk for a potentially sever response to the food allergens. However, there are limited options for patients to be exposed to specific food allergens.

### SUMMARY

In some embodiments, a food product composition includes an active ingredient comprising a food allergen protein, and one or more inactive ingredients comprising food products. The food product comprises a predefined dosage of the active ingredient.

In some embodiments, a packaged food product comprises: a food product composition as disclosed herein and packaging containing the food product.

In some embodiments, a method of treating a food allergy comprises providing a therapeutically effective amount of a food product to a subject in need thereof where the food product comprises: an active ingredient comprising a food allergen protein; and one or more non-active ingredients comprising food products. The food product comprises a predefined dosage of the active ingredient.

In some embodiments, a method of preventing a food allergy comprises: providing a therapeutically effective amount of a food product to a subject in need thereof, where the food product comprises: an active ingredient comprising a food allergen protein; and one or more non-active ingredients comprising food products. The food product comprises a predefined dosage of each active ingredient of the food allergen protein.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description:

FIG. 1 illustrates a schematic image of a food product according to some embodiments.

FIG. 2 illustrates a packaging for a food product according to some embodiments.

FIG. 3 illustrates another packaging for a food product according to some embodiments.

### DETAILED DESCRIPTION

LEAP studies have shown that the introduction of food allergens can significantly decrease the frequency of the development of food allergies and modulate the immune response to food allergens such as peanuts. This process can be referred to as early food allergen introduction or prevention to allow a patient to be proactive about food allergen prevention. When the patient has a known food allergy, the same or similar process can be used for desensitization or immunotherapy with the dosage and timing of the food allergens being directed by a doctor. Families are told to provide each food allergen early and often and told what to do. However, there are limited options for patients to ingest specific food allergens according to a prevention or therapy regimen especially in a format preferred for combining with other foods and without mixing requirements (e.g., being ready to eat). Further, there are little to no options available that combine food allergens with other healthy foods such as other non-food allergen plant proteins.

Disclosed herein is a food product that can be presented as a small, contained unit having one or more specific food allergens in a predetermined amount that can be used as part of a prevention program and/or an immunotherapy regime. The early introduction of food allergens within the body has been shown to reduce the likelihood of experiencing food allergies while also helping to provide diet diversity. Similarly, immunotherapy has been shown to have desensitization effects that may help some subjects progress towards tolerance of specific food allergens. The product can be consumed by a subject to provide a prevention regime and/or an oral immunotherapy treatment for subject that are allergic to the specific compounds and/or proteins in the food product. The product therefore provides a simple and effective way to expose a subject to the food allergen according to a number of possible regimes.

While described in some contexts herein as being used for immunotherapy, the food products described herein can also be used to desensitize a subject to various potential foods or proteins or reduce the likelihood of developing food allergies. As used herein, the term “food allergenic protein” or just “protein” refers to a protein capable of eliciting a food allergy response in a subject that is sensitive thereto. The user may be a part of a formal immunotherapy regimen or not. In other aspects, the food product described herein can be used to improve diet diversity and bring a variety of healthy foods (e.g. such as fruits, vegetables, plant-based proteins, probiotics, prebiotics, etc.) into a normal diet. Having a variety of healthy such foods in the diet can potentially improve health and avoid or reduce the formation of food allergies, eczema, and other health issues. The food product could also be used to be proactive about food allergies as part of a preventative or immunotherapy regime while helping with the gut biome to promote good health. The user may have some food allergies and want to prevent others or might not have any food allergies yet. The food product formulations can equally apply to preventative regimes, proactive diet regimes, and/or immunotherapy regimes. The disclosed food products can have the allergens split out in a ready to eat formulation that provides a convenient form for the food allergens. The food products can also comprise other non-allergen plant proteins to provide a variety of a diet.

Disclosed herein is a composition comprising a food allergen in the form of a protein having a specified dosage along with one or more inactive ingredients that serve to form a stable and cohesive food product that can be administered to a subject in need thereof. The composition can comprise a food allergen protein, one or more binders, and be in the form of a food product.

The food product can also be used as part of a preventative routine for food allergens or simply a be proactive about healthy food products. When used for prevention, one or more specific food allergens can be provided in predetermined amounts and can be taken as part of a diet to prevent the development of food allergies or sensitivities. Various exemplary forms can include butters and bars having specific food allergens include peanut proteins (e.g., 2 g of peanut proteins), cashew proteins (e.g., 2 g of cashew proteins), walnut proteins (e.g., 2 g of cashew proteins), sesame proteins (e.g., 2 g of sesame proteins), egg proteins (e.g., 2 g of egg proteins), and/or sunflower proteins (e.g., 1-2 g of sunflower proteins). The forms can include butters (e.g., proteins in an oil base) and bars, each as disclosed herein.

The active ingredient in the food product can comprise a protein of one or more food allergens. Any food allergen that can be used with a prevention plan or used with immunotherapy can be used in the food product as described herein. For example, the food allergen can be a nut protein, egg protein (e.g., baked egg proteins, boiled egg proteins, whole egg proteins, egg white proteins, etc.), dairy protein, fish protein, shellfish protein, grain protein (e.g., wheat, oat, gluten, etc.), bean proteins (e.g., soy proteins, etc.), seed proteins, legumes, peas, kiwi proteins, sunflower proteins, flax proteins, plant-based protein, or any combination thereof. The protein may correspond to the protein to which the subject is allergic or has a food sensitivity, but in some instances, cross treatments of alternative proteins can be used to treat the sensitivity to a given protein. Specific examples of proteins that can be included in the food product disclosed herein can include, but are not limited to, proteins obtained from peanuts, cashews, walnuts, pecans, almonds, brazil nuts, hazelnuts, pistachio, macadamia nuts, pine nuts, eggs, sesame seeds, yogurt, milk, probiotic yogurt, fish (e.g., salmon, white fish like code, etc.), shellfish, wheat, rye, barley, soy, pistachio, kiwi, mustard, celery, sulphites, legumes, lupin, and/or fruits. In some aspects, the proteins can be present as a defatted product. For example, peanut powder can be obtained from drying a defatted peanut, and other products can similarly be defatted prior to use of the proteins, which can be present in the solids once defatted.

The proteins can be provided in any suitable form, and the proteins may generally be provided in a shelf stable form rather than as a raw ingredient, though in some instances, a raw or minimally processed ingredient (e.g., chopped nuts, etc.) may also be used. In some aspects, the proteins can be in the form of a powder formed from the dried and/or defatted food allergen. For example, egg proteins can be provided as powdered eggs. Other food allergens such as any of the dairy allergens, fish allergens, shellfish allergens, and the like can also be provided as powders. Various grain and nut allergens can also be provided as powders (e.g., flours, etc.) once the oil or fat content has been reduced or removed. In some aspects, the proteins can be provided as a fluid such as a nut butter. A nut butter can contain the solids from the food allergen, which includes the protein, along with oils or fats contained in the nuts, all or a portion of which can be substituted for alternative fats or oils. Any other suitable forms of the proteins can also be used.

The food product can comprise any therapeutically acceptable amount of the protein. The amount of protein included in the food product can be determined by an analysis of the protein content of the active ingredients as supplied. Testing can also be performed to identify specific proteins within the overall protein content when specific proteins are being targeted for the active ingredients. In some aspects, the therapeutic amount may be at least about 1 mg, at least about 2 mg, at least about 3 mg, at least about 5 mg, at least about 10 mg, at least about 25 mg, at least about 50 mg, or at least about 100 mg, and up to about 100 grams (e.g., in a multi-serving food product), up to about 50 grams, up to about 25 grams, up to about 10 grams, or up to about 6 grams per food product.

The amount of protein per food product may be less than the amount generally prescribed for the specific protein. In some aspects, the amount of protein per food product may be between about 10 wt. % to about 100 wt. % of the prescribed amount. The ability to use a lower amount can allow the amount taken by a subject to be easily scaled up over time. For example, a patient being prescribed a dosage of 200 mg may be provided a food product having a dosage of 100 mg so that the dosage can be increased incrementally over time.

In some aspects, the food product can have a food allergen protein content of at least about 10 mg per food product, at least about 25 mg per food product, at least about 50 mg per food product, at least about 75 mg per food product, at least about 100 mg per food product, at least about 150 mg per food product, at least about 200 mg per food product, at least about 250 mg per food product, at least about 300 mg per food product, at least about 350 mg per food product, at least about 400 mg per food product, at least about 450 mg per food product, or at least about 500 mg per food product. The food product may comprise a food allergen protein content of less than about 1,000 mg per food product, less than about 900 mg per food product, less than about 800 mg per food product, less than about 700 mg per food product, or less than about 600 mg per food product. For example, each food product can have a food allergen protein content of about 100 mg, about 200 mg, about 300 mg, about 400 mg, or about 500 mg. Since the food product may be provided according to a prescribed regimen, the amount of protein within each food product may be within about 25%, within about 20%, within about 15%, within about 10%, within about 7%, within about 5%, or within about 3% of the stated weight of the food allergen protein.

Within the active ingredients, the food product can generally contain food allergen proteins for the targeted food allergen. In some instances, alternative proteins can be used to treat an allergy to a target protein. For example, pecan proteins can be used to treat walnut allergies, walnut proteins can be used to treat pecan allergies, pistachio proteins can be used to treat cashew allergies, and cashew proteins can be used to treat pistachio allergies. This form of cross treatment of allergens can also extend to other proteins. As used herein, the active ingredients can include proteins used to treat a food allergy even if the proteins are not from the source of the food allergen itself.

In some aspects, the proteins can comprise food allergens from nuts. Nut allergens are food allergens that include one or more compounds found in nuts, e.g., dry fruits that include an edible kernel or meat enclosed in a woody or leathery shell. Nut allergens of interest can include, but are not limited to, peanut allergens, (e.g., rAra h 1, rAra h 2, rAra h 3, rAra h 8 PR-10, rAra h 9 LTP, or peanut complete allergen), brazil nut allergens (e.g., rBer e 1, or brazil nut complete allergen), hazelnut or filbert allergens (e.g., rCor a 1 PR-10, rCor a 8 LTP, nCor a 9, rCor a 14, or hazelnut complete allergen), walnut allergens (e.g., rJug r 1, rJug r 3 LTP, or walnut complete allergen), cashew allergens (e.g., cashew component allergens, or cashew complete allergen), pistachio allergens (e.g., pistachio component allergens, or pistachio complete allergen), pecan allergens (e.g., pecan component allergens, or pecan complete allergen), almond allergens (e.g., almond component allergens, or almond complete allergen), or tree nut component package allergens (e.g., one or more allergens from e.g., cashew nut, walnut, hazelnut, or brazil nut).

In some aspects, the proteins can comprise food allergens from animal derived products such as dairy, eggs, shellfish, and fish. Exemplary allergens can include avian allergens (e.g., egg allergens, e.g., nGal d 1 Ovomucoid, n Gal d 2 Ovalbumin, nGal d 3 Conalbumin, or egg white complete allergen), mammalian allergens (e.g. milk allergens, e.g., nBos d 4 alpha-lactalbumin, nBos d 5 beta-lactoglobulin, nBos d 8 Casein, nBos d Lactoferrin, or milk complete allergen), or fish allergens (e.g., rCyp c 1, rGad c 1, cod complete allergen, white fish allergens, or pink fish allergens). Invertebrate animal allergens that may be present in a mixed allergen composition include crustacean allergens (e.g., shrimp allergens, e.g., rPen a 1 tropomyosin, or shrimp complete allergen).

In some aspects, the proteins can comprise food allergens that are plant based. Plant allergens can include, but are not limited to, wheat allergens (e.g., rTri a 19 Omega-5 Gliadin, gliadin wheat, rTri a 14 LTP, or wheat complete allergen), fruit allergens (e.g., kiwi allergens, e.g., rAct d 8 PR-10, or kiwi complete allergen), vegetable allergens (e.g., carrot allergens, or celery allergens, e.g., rApi g 1.01 PR-10, rPhl p 12, or celery complete allergen), legume allergens (e.g., soy allergens or chickpea allergens, e.g., rGly m 4 PR-10, nGly m 5 Beta-conglycinin, nGly m 6 Glycinin, or soy complete allergen), stone fruit allergens (e.g., f419, f420, f421, f95, f242, o214 rPru p 1 PR-10, rPru p 3 LTP, or stone fruit primary complete allergen), oat allergens (e.g., oat component allergens, or oat complete allergen), or seed allergens (e.g., sesame allergens, e.g., sesame seed component allergens, or sesame see complete allergen).

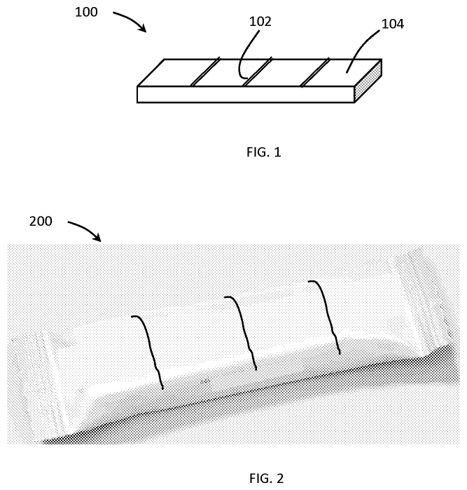

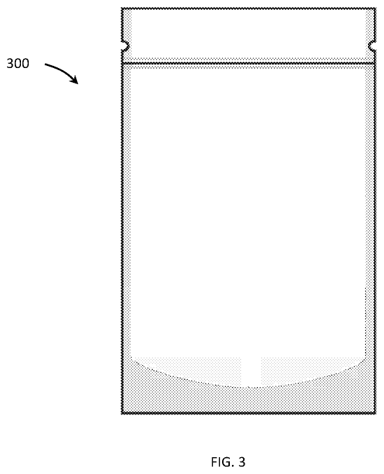

The food product can comprise one or more additional ingredients to produce a desired form such as a bar, cookie, snack, butter, nut butter, or drink. An exemplary food product 100 is illustrated in FIG. 1 in the form of a bar, though any of the forms can be used. In some instances, the food product may comprise a mixed liquid composition that contains the predetermined amount of food allergen proteins (e.g., as a butter in the packaging shown in FIG. 3, etc.). The inactive ingredients can be selected to not contain any allergen proteins of the targeted protein or other food allergens in some aspects. Further, the inactive ingredients may serve to provide the desired form, texture, and stability of the food product as well as providing one or more non-allergen plant proteins to provide food diversity.

In some aspects, various other food such as fruits, vegetables, seeds, grains, spices, sweeteners, oils, and the like can be incorporated as inactive ingredients. In some aspects, the food product can comprise one or more inactive ingredients including non-allergen plant proteins. For example, the inactive ingredients that can include non-allergen plant proteins can include, but are not limited to, mango, blueberry, strawberry, papaya, peach, pomegranate, squash (e.g., butternut squash, zucchini, etc.), carrot, sweet potatoes, spinach, broccoli, sorghum, quinoa, buckwheat, millet, amaranth, teff, oats, avocado, chia seeds, corn, parsnips, peas, beans, chickpeas, apricot, raspberries, blackberries, pineapple, dates, gojis, raisins, guava, banana, apple, kiwi, tempeh, kimchi, cherries, kiwi, or combinations thereof. Various spices can also be included such as vanilla, cinnamon, allspice or the like.

Additional additives such as oils (e.g., olive oil, avocado oil, neutral vegetable oil, canola, sunflower, corn, peanut, walnut, coconut, safflower, soy, flaxseed, sesame, margarine, butter, coconut oil, etc.), and sweeteners including sugar, and non-sugar sweeteners can also be included. In some embodiments, the food product can include a fat or oil. In some embodiments, the food composition comprises added fat. In some embodiments, the fat is saturated fat or an unsaturated fat, where the fat can be used to form a composition such as a butter.

In some aspects, the food products disclosed herein can comprise one or more additives used in food and snack products such as diluents, anti-caking agents, filling agents, fiber, enzymes (e.g., digestive enzymes, etc.), added vitamins, or the like. Any suitable additives can be used. In some aspects, the food products may comprise prebiotics. Prebiotics are compounds in foods that induce the growth or activity of beneficial microorganisms such as bacteria and fungi, which can alter the composition of organisms in the gut microbiome. Exemplary prebiotics can include oligosaccharides, galactooligosaccharides, fructooligosaccharides, oligosaccharides, inulin, or a mixture thereof.

In some aspects, the food products disclosed herein can comprise probiotics. Probiotics can include various bacterial strains such as Streptococcus thermophilus, Lactobacillus (both of which are used in the production of cow's milk yogurt) and Bifidobacterium. The probiotics can be provided as powders made by known techniques including freeze or spray drying, milling, sifting, and vibratory sifting, and/or as liquid cultures.

In some aspects, the food product may comprise only the food allergen protein and carrier, and additional food products such that the food product is free of additives such as the diluents, anti-caking agents, filling agents, etc. (e.g., organic or non-organic, non-food based products). In some aspects, the food product can be gluten and/or dairy free.

The resulting food product can take any suitable form. In some aspects, the food product can take the form of a bar. As used herein, a “bar” can comprise a food product having the active ingredients and inactive ingredients in the form of a compressed mass that forms an elongated composition. The bar can be a cooked product or an uncooked product. As an example, a bar can comprise the active ingredients within a binder formed from fruit to form a cohesive solid product. In some aspects, the bars can comprise porous or bound solids in the form of a bar such as a granola bar.

In some aspects, the food product can take the form of a cooked product. Examples of cooked products can include cookies, chips, sticks, crackers, or the like.

In some embodiments, the food product can comprise a solid form that can hold its shape within a packaging. The ability to retain a shape without crumbling or breaking may allow for the proper dosage of the food allergens to be maintained and measured by the subject. The packaging may be used to help retain the shape and cohesiveness of the food product for the subject.

In some embodiments, the food product can comprise a liquid provided within a packaging or container to hold the desired amount of food allergens with one or more additional liquids to provide the desired volume for the food product. Examples of liquid food products can comprise non-dairy milks (e.g. oat milk, etc.), juices, blends, smoothies, butters, or the like. As used herein, butters can refer to solids or other products in a base of an oil or fat, where the fat can be the same as the source of the solids or a different oil (e.g., an oil added to a different, defatted protein). Examples include peanut butter and the like. The food allergens as described herein can be present as, or formed from, a meal, flour, and/or powder, and at least some of the food allergens can initially be in a dry form (e.g., as a powder, or mix of components such as a trail mix). In some aspects, the food product can be in the form of a food allergen combined with a base product such as oatmeal.

Within the food product, the active ingredients (i.e., the food allergens) can form a portion of the overall food product while the remaining inactive ingredients can be used to provide the final form (e.g., a bar, cooked product, liquid, etc.) of the food product. In some embodiments, the active ingredients can form at least about 1 wt. %, at least about 10 wt. %, at least about 20 wt. %, or at least about 30 wt. % of the overall weight of the food product. In some aspects, the active ingredients can form up to about 98 wt. %, up to about 95 wt. %, up to about 90 wt. %, or up to about 80 wt. % of the overall weight of the food product, with the balance being inactive ingredients. The overall weight of the food product can be from about 1 gram to about 100 grams, between 5 to 60 grams, between 10 to 50 grams, or between about 10 grams and about 30 grams. As an example, the overall weight of the food product can be about 26 grams for bars and 20 grams for butters.

Each food product may be individually packaged or separated to provide a defined dosage amount. For example, each food product can be individually packaged in a sealed container. FIGS. 2 and 3 illustrate examples of containers 200, 300, respectively, for containing different forms of food products. Alternatively, a plurality of individual food products can be packaged as separated units within a single sealed package. The form of the food product may be used to prevent breakage or loss of the product within either a single package or a package with multiple food products.

In some embodiments, a single food product may comprise a plurality of doses of the active ingredient. The food product may comprise a relatively uniform distribution of the active ingredients so that the food product can be portioned into a size having the desired dosage. For example, the food product may be formed as a bar and have 2 g of a food allergen protein. As shown in FIG. 1, markings or indicators 102 on the packaging and/or the food product can be used to designate dosed portions 104 of the food product to allow for a portion with a desired dosage to be separated. When the indicators are on the food product, the indicators 102 can comprise indents, lines, press markings, or the like no the food product itself (e.g., lines no bars, indents on baked goods, etc.). When the indicators are on the packaging (e.g., packaging 200 of FIG. 2), the packaging may illustrate where each 100 mg dosed segment is on the overall food product, and the subject can then cut the bar to a size with a desired dosage. For example, the bar could be cut into a portion having 30 mg, 50 mg, 100 mg, 200 mg, 300 mg, 400 mg, 500 mg, 600 mg, 700 mg, 800 mg, or 900 mg as a dosage, or consume the entire bar for a 1000 mg dosage. The markings could be provided on the packaging, on a carrier within the packaging, or as marks or segments formed within the food product itself. In some aspects, the foot product can have 2000 mg of a food allergen protein that can be divided into 2-6 pieces to provide doses of about 1000 mg, about 650 mg, about 500 mg, about 400 mg, or about 350 mg per piece. In some aspects, the foot product can have 1200 mg of a food allergen protein that can be divided into 3-4 pieces to provide doses of about 400 mg or about 300 mg per piece.

In some aspects, a tool can be provided with the food product to cut or portion the food product. An example can include a wheeled roller to cut a bar or a sectioned press. A volume measurement such as a scoop or measurement cup (e.g., provided in a lid or other part of the packaging in some instances) can be used for food products in a liquid form. The use of a tool can provide a consistent size associated with the food product to provide a consistent dosing of the food allergen protein.

In addition to providing food allergen(s), the food products as disclosed herein can provide a diet diversity. Eating a variety of non-food allergen plant proteins can help to improve the gut biome and overall heath. It can be beneficial for children and adults to ingest a varied diet such as having 20-30 different plant proteins. To provide a variety of plant proteins, the food products can include one or more, and in some instances a plurality of, non-food allergen plant proteins. In some embodiments, the food products can comprise a base having at least 5, at least 10, at least 15, at least 20, at least 25, or at least 30 different non-allergen plant proteins, including any of those disclosed herein. There can be up to about 40 total non-allergen plant proteins within the base. The food products can then be formed as a set having a common base (e.g., with the plurality of non-allergen plant proteins) and each having one or more allergen proteins. In some aspects, the common base can be used with individual food allergen proteins. In other embodiments, one or more food products of the set can have multiple food allergen proteins combined with the base.

The food products as disclosed herein can be selected to provide a plurality of food products having at least one (or in some instances all) plant proteins that do not overlap between the food products. As an example, the food allergen lineup can include non-food allergen proteins such as blueberry proteins and spinach proteins, with the blueberry proteins present with peanut proteins, and the spinach being present with a different food allergen protein such as cashews. Subjects taking an overall line of the food products can then be supported by having a diet diversity of multiple non-food allergen plant proteins.

In some aspects, the food products can be provided as a set or group of food products. In this embodiment, a plurality of food products can be provided, each with one or more food allergens. Within the plurality of food products, the food allergens may not be repeated between the individual food products such that a given food allergen may only be present in one food product of the plurality of food products. Further, the food products can comprise one or more non-allergen plant proteins as additional ingredients. For example, each food product of the plurality of food products can comprise two or more non-allergen plant proteins in addition to the at least one food allergen. At least one of the non-allergen plant proteins may not repeated between each of the plurality of food products. For example, in a set of 5 food products, there may be at least one individual non-allergen plant protein in each of the 5 food products that is not in the other 4 food products. This can ensure that across the plurality of food products, there can be a diversity of 3-10, or between about at least 4-6 non-allergen plant proteins that can be provided as part of a diet diversity when the plurality of food products are consumed. The food allergens and the non-allergen plant proteins can include any of those described herein in any of the amounts and dosing regimes described herein.

The food products as described herein can be administered to a subject in need thereof in a therapeutically effective amount. A therapeutically effective amount is an amount of a food allergen protein needed to achieve a pharmacological effect without undue adverse side effects. The effective amount of the food allergen proteins will be selected by those skilled in the art depending on the particular subject and the disease level. It is understood that a therapeutically effective amount can vary from subject to subject, due to variation in metabolism, age, weight, general condition of the subject, the condition being treated, the severity of the condition being treated, and the judgment of the prescribing physician. In some aspects, the food products can be ingested in an amount sufficient to desensitize the subject from a food allergy. In some aspects, the food products can be ingested in order to provide a variety of plant proteins including both food allergen proteins and non-allergen plant proteins.

The methods of treating a patient may be understood by an allergist or physician, and can generally comprise administering a therapeutically effective amount in an escalating dosage regime. In some aspects, the patient can be a child, for example, the patient can be less than 6 years old, less than 5 years old, less than 4 years old, less than 3 years old, less than 2 years old, less than 18 months old, or less than 12 months old.

By way of example only, the food product can be administered to the subject for an initial period of treatment, such as for a day, multi-day period, or week. For example, a subject may be administered, 1, 2, 3, 4 or 5 doses of a food product containing a predetermined amount of food allergen proteins as described herein on in the first time period. If the subject can withstand the administered dosage, the subject may be administered a second dosage during a second time period. The second dosage can be increased during the dosage during the first time period in some aspects. The subject may continue to be administered a dosage over time to provide a desensitized response to the food allergen protein over time.

In some instances, the subject may experience adverse side effects after one or more doses. In this instance, the subject may be administered a lowered dosage during the following time period. In some instances, external factors such as an illness, injury, use of other medications, or the like may also result in a lower dosage being prescribed at a later time. In this instance, the lower dosage can be administered at a later time, and the process may be repeated and slowly increased to account for the additional factors in the administration regimen.

In addition to uses for immunotherapy, prevention, early introduction of various food proteins, desensitization, and uses for improving the immune response to food allergens, the food products as described herein can be used in some aspects for preventative applications in order to improve the diet of a subject and/or prevent the development of food allergies and other health issues over time such as eczema. The food product can comprise any of the active or inactive ingredients as described herein, and take any of the forms as described herein. The preventative food products may comprise a plurality of food allergens to provide a spectrum of exposure to the food allergens, and the food allergens may be present in higher amounts than those provided in the immunotherapy applications.

In some aspects, the food allergens can be the same as those presented above with respect to the active ingredients. Various source classes can be considered including nuts, eggs, fish, dairy, grains, beans, and similar groupings. For example, nuts may include peanuts, pecans, hazelnuts, almonds, walnuts, pistachios, and cashews. When egg proteins are used, the egg proteins in the preventative food product may comprise whole boiled egg proteins or powdered egg whites. The use of boiled egg proteins may provide a greater degree of prevention from egg allergies or other helpful related health benefits than those present in only egg white proteins or in baked egg proteins. The egg proteins may be present as a powder such as powdered whole boiled egg proteins. In some aspects, a blend of egg proteins may be used to provide a spectrum of egg proteins to help reduce the development of egg allergies or help with other related health benefits.

In some aspects, a preventative food product may comprise two or more food allergen proteins, and the food allergen proteins may be selected from different source classes. For example, the food product may comprise at least one nut protein, at least one fish protein, at least one egg protein, at least one dairy protein, at least one bean protein, and/or at least one grain protein. Specific examples can include: a nut protein with an egg protein, a nut protein with a dairy protein, a nut, fish and grain protein together, and the like. Any suitable combinations can be used to provide a preventative treatment. In some embodiments, the preventative food product may comprise at least one protein from two or more source classes.

The food allergens may be present in varying ratios. In some aspects, the food allergens within a source class may be present in relative ratios to each other between about 0.8:1 to about 1.2:1, or between about 0.9:1 to about 1.1:1, or between about 0.95:1 to about 1.05:1. In some aspects, a ratio of the food allergens between source classes may be between about 0.2:1 to about 5:1, or between about 0.4:1 to about 2.5:1, or between about 0.45:1 to about 2.2:1. The food allergen proteins may be present in amounts of between about 0.1 and about 10 g, between about 0.5 g to about 5 grams, or between about 0.8 to about 3 grams. In some aspects, nut proteins may be present in an amount of between about 0.5 g to about 4 grams, or between about 0.8 to about 1.5 grams. In some aspects, fish proteins may be present in an amount of between about 0.5 g to about 4 grams, or between about 0.8 to about 1.5 grams. In some aspects, egg proteins may be present in an amount of between about 0.5 g to about 5 grams, between about 1 to about 4 grams, or between about 1.5 to 3 grams. In some aspects, dairy proteins may be present in an amount of between about 0.5 g to about 4 grams, or between about 0.8 to about 1.5 grams. In some aspects, bean proteins (e.g. soy, etc.) may be present in an amount of between about 0.5 g to about 5 grams, or between about 0.8 to about 3 grams. In some aspects, grain proteins may be present in an amount of between about 0.5 g to about 4 grams, or between about 0.8 to about 1.5 grams. As non-limiting examples, a food product for prevention can comprise: 1) about 2 grams of peanut protein, 2) about 2 grams of egg proteins and about 1 gram of pecan protein, 3) about 1 gram of yogurt protein and about 1 gram of hazelnut protein, 4) about 1 gram of salmon protein, about 1 gram of wheat protein, and about 2 grams of almond proteins, 5) about 2 grams of sesame protein, 6) about 2 grams of walnut proteins, and/or 7) about 2 grams of cashew proteins. In some aspects, combinations of some or all of the preventative food products can be taken together.

The food products as described herein can be administered to a subject in a therapeutically effective amount as part of a preventative regime to reduce or avoid the development of food allergies. In some aspects, the subject can eat the preventative food product at least about once per week, at least about twice per week, or at least about 3 times per week in the amounts described herein in order to lessen the development of food allergies. The dosage can be adjusted over time to continue to prevent or reduce the risk of food allergies.

The food products disclosed herein can also be used to maintain a desensitization to a food allergy. In some aspects, the subject can eat the maintenance dosing to desensitize themselves to a food allergy by ingesting the food product at least about once per week, at least about twice per week, or at least about 3 times per week in the amounts described herein in order to lessen the effect (e.g., desensitization) of food allergies. The dosage can be adjusted over time to continue to maintain the desensitization to the food allergy.

The food products disclosed herein can also be used to prevent the potential development of a food allergy, and/or be proactive about food allergies and diet diversity. In some aspects, the subject can ingest the food product as described herein to maintain a diet diversity at least about once per week, at least about twice per week, or at least about 3 times per week in the amounts described herein.

### EXAMPLE

The disclosure having been generally described, the following examples are given as particular embodiments of the disclosure and to demonstrate the practice and advantages thereof. It is understood that the examples are given by way of illustration and are not intended to limit the specification or the claims in any manner.

### Example 1

As an example of a food product according to the methods and system disclosed herein, a food product useful for treating a peanut allergy can include the following ingredients: peanut butter, defatted peanut flower, quinoa, blueberry powder, spinach powder, and banana powder. The food product can be formed into a bar having a measured amount of peanut proteins, and the bar can be provided to a subject in need thereof according to an oral dosage regime to lessen or alleviate the symptoms of (e.g., desensitize, etc.) a peanut allergy. The bar can also be used for the prevention of food allergies or a health snack or diet diversity.

### Example 2

As an example of a food product according to the methods and system disclosed herein, a food product useful for treating a peanut allergy can include the following ingredients: peanut butter, defatted peanut flower, peanut oil or sunflower oil, blueberry powder, and vanilla. The food product can be formed into a butter or paste that can be provided to a subject in need thereof according to an oral dosage regime to lessen or alleviate the symptoms of (e.g., desensitize, etc.) a peanut allergy. The bar can also be used for the prevention of food allergies or a health snack or diet diversity.

### Example 3

As an example of a food product according to the methods and system disclosed herein, a food product useful for treating a sesame allergy can include the following ingredients: seeds, legumes, tahini (e.g., a sesame seed butter), defatted sesame powder, freeze dried banana powder, vanilla, and olive oil. The food product can be formed into a butter or paste that can be provided to a subject in need thereof according to an oral dosage regime to lessen or alleviate the symptoms of (e.g., desensitize, etc.) a sesame allergy. The bar can also be used for the prevention of food allergies or a health snack or diet diversity.

### Example 4

As an example of a food product according to the methods and system disclosed herein, a food product useful for treating a cashew allergy can include the following ingredients: cashew butter, freeze dried mango powder, vanilla, and olive oil. The food product can be formed into a butter or paste that can be provided to a subject in need thereof according to an oral dosage regime to lessen or alleviate the symptoms of (e.g., desensitize, etc.) cashew peanut allergy. The bar can also be used for the prevention of food allergies or a health snack or diet diversity.

### Example 5

As an example of a food product according to the methods and system disclosed herein, a food product useful for treating a walnut allergy can include the following ingredients: walnut butter, freeze dried apple powder, vanilla, and olive oil. The food product can be formed into a butter or paste that can be provided to a subject in need thereof according to an oral dosage regime to lessen or alleviate the symptoms of (e.g., desensitize, etc.) a walnut allergy. The bar can also be used for the prevention of food allergies or a health snack or diet diversity.

### Example 6

As an example of a food product according to the methods and system disclosed herein, a food product useful for treating an egg allergy can include the following ingredients: egg powder, sunflower butter, sunflower oil, and pineapple. The food product can be formed into a butter or paste that can be provided to a subject in need thereof according to an oral dosage regime to lessen or alleviate the symptoms of (e.g., desensitize, etc.) an allergy. The bar can also be used for the prevention of food allergies or a health snack or diet diversity.

Having described various food products, methods of treating a subject, and processes for preparing the food products, certain aspects can include, but are not limited to:

In a first aspect, a food product composition comprises: an active ingredient comprising a food allergen protein; and one or more inactive ingredients comprising food products, wherein the food product comprises a predefined dosage of the active ingredient.

A second aspect can include the food product of the first aspect, wherein the food product comprises a food allergen protein formed from at least one of: a nut protein, egg protein, dairy protein, fish protein, shellfish protein, grain protein, soy proteins, plant based protein, or any combination thereof.

A third aspect can include the food product of the first aspect, wherein the food allergen protein is derived from at least one of: peanuts, cashews, walnuts, pecans, almonds, brazil nuts, hazelnuts, macadamia nuts, pine nuts, eggs, sesame seeds, yogurt, milk, probiotic yogurt, fish (e.g., salmon, white fish like code, etc.), shellfish, wheat, rye, barley, soy, fruits, pistachio, seeds, legumes, peas, kiwi, sunflower, flax, mustard, celery, sulphites, legumes, lupin, or any combination thereof.

A fourth aspect can include the food product of any one of the first to third aspects, wherein the food allergen protein is in the form of a powder, butter, raw ingredients, a drink, a bar, or any combination thereof.

A fifth aspect can include the food product of any one of the first to fourth aspects, wherein the food allergen protein can have a weight between about 1 mg to about 10 g in the food product.

A sixth aspect can include the food product of any one of the first to fifth aspects, wherein only a single food allergen protein is present in the food product.

A seventh aspect can include the food product of any one of the first to sixth aspects, wherein the inactive ingredient comprises fruits, vegetables, seeds, grains, spices, sweeteners, oils, or any combination thereof.

An eighth aspect can include the food product of any one of the first to seventh aspects, wherein the inactive ingredients comprise diluents, anti-caking agents, filling agents, or combinations thereof.

A ninth aspect can include the food product of any one of the first to seventh aspects, wherein the food product is free of diluents, anti-caking agents, filling agents, or combinations thereof.

A tenth aspect can include the food product of any one of the first to ninth aspects, wherein the food product is in the form of a bar, a cooked product, a butter, or a liquid.

In an eleventh aspect, a packaged food product comprises: one or more of the food products of any one of the first to tenth aspects; and packaging containing the food product.

An eleventh aspect can include the packaged food product of the eleventh aspect, wherein the food product is individually disposed in the packaging.

A thirteenth aspect can include the packaged food product of the eleventh aspect, wherein a plurality of the food product is disposed in the packaging.

A fourteenth aspect can include the packaged food product of the eleventh aspect, wherein the packaging comprises dosage indicators corresponding to defined dosages of the food product.

A fifteenth aspect can include the packaged food product of the eleventh aspect, wherein the food product comprises dosage indicators corresponding to defined dosages of the food product.

In a sixteenth aspect, a method of treating a food allergy comprises: providing a therapeutically effective amount of a food product to a subject in need thereof, wherein the food product comprises: an active ingredient comprising a food allergen protein; and one or more non-active ingredients comprising food products, wherein the food product comprises a predefined dosage of the active ingredient.

A seventeenth aspect can include the food product of the sixteenth aspect, wherein the food product comprises a food allergen protein formed from at least one of: a nut protein, egg protein, dairy protein, fish protein, shellfish protein, grain protein, soy protein, plant based protein seeds, legumes, peas, kiwi, sunflower, flax, or any combination thereof.

An eighteenth aspect can include the food product of the sixteenth aspect, wherein the food allergen protein is derived from at least one of: peanuts, cashews, walnuts, pecans, almonds, brazil nuts, hazelnuts, macadamia nuts, pine nuts, eggs, sesame seeds, yogurt, milk, probiotic yogurt, fish (e.g., salmon, white fish like code, etc.), shellfish, wheat, rye, barley, soy, fruits, pistachio, kiwi, mustard, celery, sulphites, legumes, lupin, or any combination thereof.

A nineteenth aspect can include the food product of any one of the sixteenth to eighteenth aspects, wherein the food allergen protein is in the form of a powder, butter, raw ingredient, or any combination thereof.

A twentieth aspect can include the food product of any one of the sixteenth to nineteenth aspects, wherein each of the food allergen protein can have a weight between about 1 mg to about 10 g in the food product.

A twenty first aspect can include the food product of any one of the sixteenth to twentieth aspects, wherein only a single food allergen protein is present in the food product.

A twenty second aspect can include the food product of any one of the sixteenth to twenty first aspects, wherein the inactive ingredient comprises fruits, vegetables, seeds, grains, spices, sweeteners, oils, or any combination thereof.

A twenty third aspect can include the food product of any one of the sixteenth to twenty second aspects, wherein the inactive ingredients comprise diluents, anti-caking agents, filling agents, or combinations thereof.

A twenty fourth aspect can include the food product of any one of the sixteenth to twenty second aspects, wherein the food product is free of diluents, anti-caking agents, filling agents, or combinations thereof.

A twenty fifth aspect can include the food product of any one of the sixteenth to twenty fourth aspects, wherein the food product is in the form of a bar, a cooked product, a butter, or a liquid.

In a twenty sixth aspect, a method of preventing a food allergy comprises: providing a therapeutically effective amount of a food product to a subject in need thereof, wherein the food product comprises: an active ingredient comprising a plurality of allergen proteins; one or more non-active ingredients comprising food products, wherein the food product comprises a predefined dosage of each active ingredient of the plurality of food allergen proteins. The food product can also be used for desensitization or as part of proactively providing a diet diversity, which can increase the gut biome and improve health.

A twenty seventh aspect can include the food product of the twenty sixth aspect, wherein the food product comprises at least two food allergen proteins formed from at least one of: a nut protein, egg protein, dairy protein, fish protein, shellfish protein, grain protein, soy protein, plant based protein or any combination thereof.

A twenty eighth aspect can include the food product of the twenty sixth aspect, wherein the plurality of food allergen proteins is derived from at least one of: peanuts, cashews, walnuts, pecans, almonds, brazil nuts, hazelnuts, macadamia nuts, pine nuts, eggs, sesame seeds, yogurt, milk, probiotic yogurt, fish (e.g., salmon, white fish like code, etc.), shellfish, wheat, rye, barley, soy, fruits, pistachio, kiwi, mustard, celery, sulphites, legumes, lupin, or any combination thereof.

A twenty ninth aspect can include the food product of any one of the twenty sixth to twenty eighth aspects, wherein the plurality of food allergen proteins is in the form of a powder, butter, raw ingredient, or any combination thereof.

A thirtieth aspect can include the food product of any one of the twenty sixth to twenty ninth aspects, wherein the food allergen protein can have a weight between about 1 mg to about 10 g in the food product.

A thirty first aspect can include the food product of any one of the twenty sixth to thirtieth aspects, wherein the inactive ingredient comprises fruits, vegetables, seeds, grains, spices, sweeteners, oils, or any combination thereof.

A thirty second aspect can include the food product of any one of the twenty sixth to thirty first aspects, wherein the inactive ingredients comprise diluents, anti-caking agents, filling agents, or combinations thereof.

A thirty third aspect can include the food product of any one of the twenty sixth to thirty second aspects, wherein the food product is free of diluents, anti-caking agents, filling agents, or combinations thereof.

A thirty fourth aspect can include the food product of any one of the twenty sixth to thirty third aspects, wherein the food product is in the form of a bar, a cooked product, a butter, or a liquid.

A thirty fifth aspect can include the food product of any one of the twenty sixth to thirty fourth aspects, wherein the food product comprises at least one nut allergen and at least one egg allergen, and wherein a weight ratio of the nut allergen to the egg allergen is between 0.2:1 to about 5:1.

A thirty sixth aspect can include the food product of the thirty fifth aspect, wherein the food product comprises at least about 0.5 g of each of the plurality of food allergen proteins.

It is to be further understood that the present description is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present systems and methods. It must be noted that as used herein and in the appended claims (in this application, or any derived applications thereof), the singular forms “a,” “an,” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. As used herein, the term “about” with respect to a range or value can include values within 20% (e.g., plus or minus) of the stated value. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this description belongs. Preferred methods, techniques, devices, and materials are described, although any methods, techniques, devices, or materials similar or equivalent to those described herein may be used in the practice or testing of the present systems and methods. Structures described herein are to be understood also to refer to functional equivalents of such structures. The present systems and methods will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although claims may be formulated in this application or of any further application derived therefrom, to particular combinations of features, it should be understood that the scope of the disclosure also includes any novel feature or any novel combination of features disclosed herein either explicitly or implicitly or any generalization thereof, whether or not it relates to the same systems or methods as presently claimed in any claim and whether or not it mitigates any or all of the same technical problems as do the present systems and methods.

Features which are described in the context of separate embodiments may also be provided in combination in a single embodiment. Conversely, various features which are, for brevity, described in the context of a single embodiment, may also be provided separately or in any suitable sub-combination. The Applicant(s) hereby give notice that new claims may be formulated to such features and/or combinations of such features during the prosecution of the present Application or of any further Application derived therefrom.

What is claimed is:

1. A food product composition comprising:
an active ingredient comprising a food allergen protein;
one or more inactive ingredients comprising food-based ingredients,
wherein the food product requires no mixing prior to consumption,
wherein the food product is substantially free of anti-caking agents and filling agents,
wherein the food product comprises a predefined dosage of the active ingredient, and
wherein the predefined dosage is within about 20% of a stated weight of the food allergen protein.

2. The food product composition of claim 1, wherein the food allergen protein is derived from at least one of: peanuts, cashews, walnuts, pecans, almonds, brazil nuts, hazelnuts, macadamia nuts, pine nuts, eggs, sesame seeds, milk, fish, shellfish, wheat, rye, barley, soy, kiwi, sunflower, flax, legumes, lupin, or any combination thereof.

3. The food product composition of claim 1, wherein the one or more inactive ingredients comprise one or more of a fruit, a vegetable, a seed, a grain, a spice, a sweetener, or an oil.

4. The food product composition of claim 1, wherein the one or more inactive ingredients further comprise one or more non-allergen plant proteins selected to provide diet diversity to a subject.

5. The food product composition of claim 4, wherein the one or more inactive ingredients comprise at least 5 different non-allergen plant proteins.

6. The food product composition of claim 1, wherein the food product is in the form of a bar comprising a compressed mass forming an elongated composition that retains its shape within a packaging.

7. The food product composition of claim 1, wherein the food product is in the form of a butter comprising defatted protein solids in a base of an oil or fat.

8. The food product composition of claim 1, wherein the food product is in the form of a cooked product selected from cookies, chips, sticks, and crackers.

9. The food product composition of claim 1, wherein the food product has an overall weight of between about 10 grams and about 30 grams.

10. The food product composition of claim 1, wherein the food allergen protein comprises at least about 10 wt. % of the overall weight of the food product.

11. The food product composition of claim 1, wherein the food product is free of intentionally added emulsifiers, stabilizers, and viscosifiers.

12. The food product composition of claim 1, wherein the food product comprises dosage indicators on the food product comprising indents, lines, or press markings corresponding to defined dosages of the active ingredient.

13. The food product composition of claim 12, wherein the food product comprises a plurality of predefined doses of the active ingredient and is divisible along the dosage indicators to provide a single predefined dose per portion.

14. A method of treating or preventing a food allergy in a subject comprising administering to the subject a therapeutically effective amount of a food product composition comprising a food allergen protein as an active ingredient and one or more food-based ingredients as inactive ingredients, wherein the food product composition requires no mixing prior to consumption and comprises a predefined dosage of the food allergen protein.

15. The method of claim 14, wherein the subject is less than 6 years of age.

16. The method of claim 14, wherein the subject has been diagnosed with a food allergy.

17. The method of claim 14, wherein the food allergen protein is administered in an escalating dosage regimen comprising at least a first dosage administered during a first time period and a second dosage that is greater than the first dosage administered during a second time period.

18. The method of claim 14, wherein the food product composition is administered at least about once per week to maintain a desensitization to a food allergy in a subject who has previously undergone allergen immunotherapy.

19. The method of claim 14, wherein the subject has not been diagnosed with a food allergy and the food product composition is administered to prevent development of a food allergy or to provide diet diversity.

20. A kit for oral allergen immunotherapy or allergy prevention comprising:
a plurality of food product compositions each according to claim 1, wherein the food product compositions comprise different predefined dosages of the food allergen protein arranged in an escalating order for sequential administration to a subject.
